(19) United States
(12) Patent Application Publication
(10) Pub. No.: US 2013/0317190 A1
(43) Pub. Date: Nov. 28, 2013

(54) COPOLYMERIZABLE SURFACTANTS

(71) Applicants: Thomas Schliwka, Bergisch Gladbach (DE); Thomas Mausberg, Haan (DE); Uwe Held, Velbert (DE); Ronald Klagge, Erkrath (DE); Stefan Busch, Dusseldorf (DE); Markus Scherer, Koln (DE)

(72) Inventors: Thomas Schliwka, Bergisch Gladbach (DE); Thomas Mausberg, Haan (DE); Uwe Held, Velbert (DE); Ronald Klagge, Erkrath (DE); Stefan Busch, Dusseldorf (DE); Markus Scherer, Koln (DE)

(73) Assignee: Cognis IP Management GmbH, Dusseldorf (DE)

(21) Appl. No.: 13/979,770

(22) PCT Filed: Oct. 8, 2011
(86) PCT No.: PCT/EP11/05040

§ 371 Date: Jul. 15, 2013

(30) Foreign Application Priority Data
Jan. 18, 2011 (EP) EP11151299

(51) Int. Cl. C07D 303/48; C08F 224/00; C07F 9/655

(52) U.S. Cl. CPC C07D 303/48; C07F 9/65502; C08F 224/00
USPC 526/229; 549/512; 549/217; 526/273

(57) ABSTRACT

The invention relates to the use of compounds of the formula (I), R—CH(O—(AO)ₘX)—CH₂—O—(AO)—CH₂—CH═CH₂, where: R denotes an alkyl radical which has 8 to 18 carbon atoms and can be saturated or unsaturated, straight-chain or branched; X denotes a sulfate or phosphate group or hydrogen; (AO) denotes an alkylene oxide unit, selected from the group consisting of ethylene oxide, propylene oxide or butylene oxide; n denotes a number in the range of 0 to 50; and m denotes zero or a number in the range of 1 to 30; as copolymerizable emulsifiers in the emulsion polymerization of olefinically unsaturated monomers.

### FIELD OF THE INVENTION

The present invention lies within the polymer sector and relates to allyl ethers with a specific structure and also to their use as emulsifiers in emulsion polymerization and also to a process for preparing polymers by emulsion polymerization using the allyl ethers of the present invention, which are advantageous copolymerizable emulsifiers.

### STATE OF THE ART

Emulsion polymerization is a specific method of polymerization wherein olefinically unsaturated monomers of low solubility in water are emulsified in water by means of emulsifiers and polymerized using water-soluble initiators such as potassium peroxodisulfate or redox initiators for example. Anionic and/or nonionic surfactants are the key constituents here in that they ensure the process of emulsion polymerization via micelle construction in the aqueous solution.

Copolymerizable emulsifiers are in great demand in industry since they are wholly or partly incorporated in the growing polymer chain and thus reduce the migration of free emulsifier molecules in the end-use product for example. Copolymerizable emulsifiers occupy a position halfway between monomers and conventional emulsifiers. Their reactivity has to be tailored to the monomer system used and they must not have an adverse effect on the properties of the polymer formed. At the same time, they must not lose their emulsificative properties as a result of the presence of a reactive group. Owing to this combination of special properties, novel copolymerizable emulsifiers are greatly sought after in industry.

German laid-open specification DE-A-10340081 describes copolymerizable surfactants of the formula HOOC—CH═CH—COO—(BO)_{z}(PO)_{y}(EO)ₓR¹ where R¹ is an alkyl radical or alkylphenol radical having 8 to 24 carbon atoms, BO is a butylene oxide unit, PO is a propylene oxide unit and EO is an ethylene oxide unit, and the numbers x, y and z are each independently 0 or numbers from 1 to 50, with the proviso that at least one of x, y and z is other than 0, wherein the carboxyl group may be wholly or partly present in neutralized form and the C═C double bond may be cis- or trans-configured.

EP-A-1,825,908 describes specific compounds of structure (A)

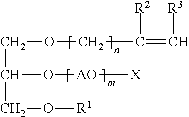

where R¹ is a C₈₋₂₀ hydrocarbyl group, R² and R³ are each hydrogen or a methyl group, (AO) is a C₂₋₄ alkylene oxide group, X is an ionic hydrophilic group, n is from 0 to 12 and m is from 0 to 100. These compounds (A) are used in combination with certain nitrogen compounds (B) and the specific composition comprising (A) and (B) is said to be useful as an emulsifier in emulsion polymerization.

### DESCRIPTION OF THE INVENTION

The problem addressed by the present invention is that of providing compounds which, singly or admixed with other compounds, are useful as copolymerizable emulsifiers for emulsion polymerization. Used as emulsifiers for emulsion polymerization, these should have the particular effect of minimizing coagulum.

Furthermore, these copolymerizable emulsifiers should be pourable/pumpable in an aqueous offering.

Finally, the use as emulsifiers in emulsion polymerization should provide latices which, compared with latices obtained using comparable non-copolymerizable emulsifiers, have improved properties with regard to the water resistance and scratch resistance of polymer films obtained therefrom.

The present invention first provides compounds of general formula (I),

R—CH(O—(AO)ₘX)—CH₂—O—(AO)ₙ—CH₂—CH═CH₂ (I)

where
R is an alkyl radical of 8 to 18 carbon atoms, which may be saturated or unsaturated, straight chain or branched,
X is a sulfate or phosphate group or hydrogen,
(AO) is an alkylene oxide unit selected from the group consisting of ethylene oxide, propylene oxide and butylene oxide,
n is from 0 to 50, and
m is zero or from 1 to 30.

The present invention further provides for the use of compounds of general formula (I),

R—CH(O—(AO)ₘX)—CH₂—O—(AO)ₙ—CH₂—CH═CH₂ (I)

where
R is an alkyl radical of 8 to 18 carbon atoms, which may be saturated or unsaturated, straight chain or branched,
X is a sulfate or phosphate group or hydrogen,
(AO) is an alkylene oxide unit selected from the group consisting of ethylene oxide, propylene oxide and butylene oxide,
n is from 0 to 50, and
m is zero or from 1 to 30,
   as a copolymerizable emulsifier in the emulsion polymerization of an olefinically unsaturated monomer. In this emulsion polymerization, said compounds (I) can be used individually or mixed with each or one another—and also combined with conventional emulsifiers, if desired.

In said formula (I), the building block (AO) is an alkylene oxide unit selected from the group consisting of ethylene oxide, propylene oxide and butylene oxide. The indices n and m in said formula (I) denote in connection with the structural building blocks (AO)ₙ and (AO)ₘ the average number of alkylene oxide building blocks.

Said compounds (I) are obtainable by any method known to a person skilled in the art. This typically takes the form of reacting alpha-olefin epoxides with addition products of ethylene oxide, propylene oxide and/or butylene oxide onto allyl alcohol by opening the oxirane ring of the epoxides. The OH group formed in the course of ring opening can be converted into a sulfate or phosphate group, if desired; this conversion into a sulfate or phosphate group can be preceded by an alkoxylation, if desired.

Addition products of ethylene oxide or propylene oxide and/or butylene oxide onto allyl alcohol, as will be known to a person skilled in the art, are obtainable by reacting allyl alcohol with ethylene oxide, propylene oxide and/or butylene oxide in the presence of an alkoxylation catalyst; this may be illustrated using the following formula scheme, where (AO) and n have the same meanings as in formula (I):

CH₂═CH—CH₂—OH+n(AO)→CH₂═CH—CH₂—O—(AO)ₙ—H

The alkoxylation can be carried out using ethylene oxide, propylene oxide and butylene oxide individually or mixed with each or one another. The index n is a (statistical) mean; so in the case of CH₂═CH—CH₂—O—(AO)ₙ—H the statement n=1 has the meaning that 1 mol of allyl alcohol has been reacted with 1 mol of alkylene oxide, while n=2 has the meaning that 1 mol of allyl alcohol has been reacted with 2 mol of alkylene oxide, n=6 has the meaning that 1 mol of allyl alcohol has been reacted with 6 mol of alkylene oxide, and so on. Hence the index n represents the molar reaction ratio of allyl alcohol and employed alkylene oxide, while the allyl alcohol alkoxylates can differ in their homolog distribution depending on the catalyst used.

The index n in formula (I), as mentioned, is a number from 0 to 50. Preferably it is from 1 to 30 and especially from 3 to 10. Since n designates the number of alkylene oxide units (AO)ₙ and the alkylene oxide units are selected from the group consisting of ethylene oxide, propylene oxide and butylene oxide, it may be expressly noted that this is to be understood as meaning that n denotes the sum total of all ethylene oxide, propylene oxide and butylene oxide units present in the entire (AO)ₙ segment (which is to be understood as meaning the entirety represented by (AO)ₙ). The entire (AO)ₙ segment can be constructed exclusively of ethylene oxide, propylene oxide or butylene oxide units or contain these units in mixed form—as random distribution or in block. Preferably, the entire (AO)ₙ segment of compounds (I) is constructed exclusively of ethylene oxide and/or propylene oxide units. More particularly, the entire (AO)ₙ segment of compounds (I) is constructed exclusively of ethylene oxide units.

The index m in formula (I), as mentioned, is zero or a number from 1 to 30. In one embodiment, m is zero. In a further embodiment, m is from 1 to 10 and especially from 3 to 10. Since m designates the number of alkylene oxide units (AO)ₘ and the alkylene oxide units are selected from the group consisting of ethylene oxide, propylene oxide and butylene oxide, it may be expressly noted that this is to be understood as meaning that m denotes the sum total of all ethylene oxide, propylene oxide and butylene oxide units present in the entire (AO)ₘ segment (which is to be understood as meaning the entirety represented by (AO)ₘ). The entire (AO)ₘ segment can be constructed exclusively of ethylene oxide, propylene oxide or butylene oxide units or contain these units in mixed form—as random distribution or in block. Preferably, the entire (AO)ₘ segment of compounds (I) is constructed exclusively of ethylene oxide and/or propylene oxide units. More particularly, the entire (AO)ₘ segment of compounds (I) is constructed exclusively of ethylene oxide units.

The group X is a sulfate or phosphate group or hydrogen.

In one embodiment, the group X is hydrogen.

In one embodiment, the group X is a sulfate or phosphate group. The sulfate and/or phosphate groups X in compounds (I) are in partly or wholly neutralized form. The sulfate or phosphate group may be neutralized, for example, with alkali or alkaline earth metal hydroxides such as sodium hydroxide, potassium hydroxide, calcium hydroxide or magnesium hydroxide or with amines such as ammonia or ethanolamines. The salt form of compounds (I) is notable for good solubility in water.

The compounds of formula (I) are obtainable, for example, by reacting an alkoxylated allyl alcohol with an alpha-olefin oxide and, if desired, sulfating or phosphating the product obtained (where X=H).

Compounds (I) to be used according to the present invention polymerize readily and completely together with other olefinically unsaturated monomers different therefrom while promoting the formation of a foam-free and homogeneous emulsion.

Compounds (I) where X is a sulfate or phosphate group are preferred. They are preferably used in the emulsion polymerization in partly or wholly neutralized form (“salt form” of the sulfate or phosphate group). This partly or wholly neutralized form is readily obtainable by partly or wholly neutralizing said compounds (I) in a conventional manner, for example with alkali or alkaline earth metal hydroxides such as sodium hydroxide, potassium hydroxide, calcium hydroxide or magnesium hydroxide or with amines such as ammonia or ethanolamines. The salt form of compounds (I) is notable for good solubility in water.

The present invention further provides a process for preparing polymers by emulsion polymerization of olefinically unsaturated monomers wherein said abovementioned compounds (I) are used as copolymerizable emulsifiers.

The present invention process using compounds (I), particularly in salt form, provides polymers having special shear and electrolyte stability and also a low coagulum content.

One embodiment of the invention provides latices which in turn are additionally notable for stability to temperature fluctuations and which do not give rise to any discernible migration of the emulsifier into the film.

There is a further advantage to the process of the present invention in that it is virtually foam-free and reliably avoids the formation of volatile organic compounds. Since incorporation of emulsifier (I) in the polymer is virtually quantitative, the use thereof also does not present any biodegradability issues. The olefinically unsaturated compounds (I) are further virtually devoid of any tendency to homopolymerize.

It was found that the use of compounds (I) as emulsifiers in emulsion polymerization provides latices having improved properties in respect of water resistance and scratch resistance on the part of polymer films obtained therefrom compared with such latices obtained with comparable non-copolymerizable emulsifiers. Combinations of compounds (I) with nonionic or anionic surfactants other than (I) can also be used, and likewise exhibit a positive profile of properties.

The latices of the present invention can be used in the coatings industry for example. It was found that coatings obtained using the latices of the present invention possess higher corrosion protection than conventional coatings.

It was further found that notably latices obtained using compounds of general formula (I) as emulsifiers have an improved freeze-thaw stability over those latices obtained with conventional emulsifiers.

### Monomers

The olefinically unsaturated esters of the general formula (I) to be used according to the present invention are useful as emulsifiers in the emulsion polymerization of virtually all industrially important, substantially water-insoluble monomers, but preferably (meth)acrylic, styrenic and vinylic compounds.

Typical examples of these monomers are vinylaromatics, e.g., styrene, divinylbenzene or vinyltoluene, polymerizable olefins and diolefins such as propene, butadiene or isoprene, esters of acrylic or methacrylic acid with linear or branched alcohols having 1 to 18 carbon atoms, more particularly of alcohols having 1 to carbon atoms and—particularly preferably—of methyl esters, ethyl esters and butyl esters thereof, vinyl esters of acids having 2 to 12 carbon atoms, more particularly vinyl acetate, vinyl propionate, vinyl 2-ethylhexanoate and vinyl laurate, vinyl alkyl ethers having alkyl groups of 1 to 8 carbon atoms, vinyl chloride, vinylidene chloride and the like.

Monomers selected from the group of alkyl acrylates, styrene acrylates, VeoVa compounds or mixtures thereof, with or without addition of acrylic acid or methacrylic acid, are particularly preferred in the context of the present invention.

In the presence of the copolymerizable emulsifiers (I) to be used according to the present invention, the monomers can be homopolymerized or they can be copolymerized with others of the recited compounds from the above listing. It is further possible to perform copolymerizations involving up to 50% by weight of further inherently partly or wholly water-soluble monomers other than the compounds (I) according to the present invention, examples being acrylonitrile, methacrylonitrile, monoesters of maleic and/or fumaric acid with 1 to 8 carbon atoms, acrylic acid, methacrylic acid, maleic acid, fumaric acid, crotonic acid and/or itaconic acid.

In one embodiment, monomers used in the process of the present invention are combinations of styrene/butyl acrylate, vinyl acetate/butyl acrylate or styrene/butadiene.

### Co-emulsifiers

It is further also possible for the compounds (I) and (II) which are to be used according to the present invention to be used in combination with known nonionic and/or anionic co-emulsifiers. This can lead to dispersions of enhanced stability, for example in respect of shearing forces, temperature effects and electrolytes. The co-emulsifiers are added in amounts of 0.5% to 5% and preferably 1% to 3% by weight, based on total monomers used. The co-emulsifiers may be initially charged at the start of the polymerization together with the emulsifiers, or may be added in the course of the polymerization. In a further version, a pre-emulsion is prepared using or co-using the co-emulsifiers and added in the course of the polymerization. It is also possible for the dispersions obtained using the acrylic and/or methacrylic esters of the present invention to be admixed with co-emulsifiers for post-stabilization.

The compounds (I) to be used according to the present invention can also be used together with protective colloids. Typical examples of protective colloids of this type are fully or partially hydrolyzed homo- and/or copolymers of vinyl acetate, e.g., partially hydrolyzed polyvinyl acetate, or fully hydrolyzed copolymers of vinyl acetate and vinyl ethers. Preferred copolymers have from 1 to 4 carbon atoms in the ether moiety of the polyvinyl ether. Further protective colloids may be derived from polysaccharides. Especially cellulose ethers such as hydroxyethylcellulose, hydroxypropylcellulose, carboxymethylcellulose, methylcellulose, ethylcellulose or cellulose mixed ethers are suitable. Polyacrylamide and its copolymers with acrylic acid, acrylonitrile or acrylic esters are also suitable. It is similarly possible to use condensation products of naphthalenesulfonic acid and formaldehyde or other water-soluble formaldehyde resins, particularly urea-formaldehyde resins. Finally casein, gelatin, gum arabic and also natural starch and substituted derivatives of starch such as hydroxyethyl starch are suitable protective colloids.

### Emulsion Polymerization

One embodiment utilizes the emulsifiers (I) in emulsion polymerization in amounts from 0.1% to 25% by weight, based on the sum total of the monomers.

The aqueous dispersions typically to be prepared in the first step of the process using compounds (I) include in practice from 15% to 75% by weight of polymerized monomers (dry residue) in water or a mixture of water and water-soluble organic solvents. The range from 20% to 60% by weight of dry residue is preferred; however, aqueous dispersions with less than 15% by weight of dry residue are also obtainable for special applications. The aforementioned processes of emulsion polymerization may also utilize further customary polymerization aids, more particularly initiators, for example inorganic peroxide compounds such as potassium persulfate or ammonium persulfate or hydrogen peroxide; organic peroxide compounds or organic azo compounds, where these can be used for emulsion polymerization.

Initiators are used in customary amounts, i.e., from 0.05% to 2% by weight and preferably from 0.1% to 0.5% by weight. Suitable aids further include buffer substances, e.g., sodium bicarbonate, sodium pyrophosphate or sodium acetate, which can each be used in amounts of up to 2% by weight. Accelerants such as formaldehydesulfoxylate can also be used. It is further possible to use customary molecular weight regulators used in emulsion polymerization, e.g., butenol or else organic thio compounds such as mercaptoethanol, thioglycolic acid, octyl mercaptan or tert-dodecyl mercaptan. To perform the polymerization processes various methods typically employed in emulsion polymerization are possible, for example a total initial charge of all the reactants, a monomer feed or an emulsion feed. In general, the temperature of the polymerization medium is maintained for this purpose in a range from 40 to 100 and more particularly from 50 to 90° C. The pH is advantageously maintained in a range between 3 and 9, although the compounds of the present invention also tolerate an emulsion polymerization at lower pH values. The aforementioned possible versions of the emulsion polymerization process are advantageously carried out in coolable and heatable containers equipped with stirrers and temperature measurement equipment, for example stirred pressure tanks. It is similarly possible to use coiled-tube reactors or so-called loop reactors. After the polymerization has ended, the polymer dispersion is advantageously cooled down and removed from the reactor via screening means. When the reaction products are to be isolated as solid products, the polymer dispersion is advantageously precipitated or spray dried. Preferably, however, the as-polymerized dispersions are used directly as binders for paints, adhesives, papercoating slips or other coating materials. Further conditions for emulsion polymerization processes using the compounds (I) which are to be used according to the present invention can be freely chosen or adapted to the particular requirements in a conventional manner by a person skilled in the art.

1. A compound of formula (I),
R—CH(O—(AO)ₘX)—CH₂—O—(AO)ₙ—CH₂—CH═CH₂ (I)
where
R is an alkyl radical of 8 to 18 carbon atoms, which may be saturated or unsaturated, straight chain or branched,
X is a sulfate or phosphate group or hydrogen,
(AO) is an alkylene oxide unit selected from the group consisting of ethylene oxide, propylene oxide and butylene oxide,
n is from 0 to 50, and
m is zero or from 1 to 30.

2. A method of preparing a polymer, the method comprising emulsifying an olefinically unsaturated monomer in water with a copolymerizable emulsifier comprising a compound of formula (I),
R—CH(O—(AO)ₘX)—CH₂—O—(AO)ₙ—CH₂—CH═CH₂ (I)
where
R is an alkyl radical of 8 to 18 carbon atoms, which may be saturated or unsaturated, straight chain or branched,
X is a sulfate or phosphate group or hydrogen,
(AO) is an alkylene oxide unit selected from the group consisting of ethylene oxide, propylene oxide and butylene oxide,
n is from 0 to 50, and
m is zero or from 1 to 30,
during emulsion polymerization of the olefinically unsaturated monomer.

3. The method of claim 2, wherein X is a sulfate or phosphate group.

4. The method of claim 3, wherein the sulfate or phosphate group is in partly or wholly neutralized form.

5. The method of claim 2, wherein the degree of alkoxylation n is from 1 to 30.

6. The method of claim 5, wherein the degree of alkoxylation n is from 3 to 10 and (AO) is an ethylene oxide unit.

7. A process for preparing a polymer by emulsion polymerization of an olefinically unsaturated monomer, which process comprises utilizing said compound (I) according to claim 1 as a copolymerizable emulsifier.

8. The compound of claim 1, wherein X is a sulfate or phosphate group.

9. The compound of claim 8, wherein the sulfate or phosphate group is in partly or wholly neutralized form.

10. The compound of claim 1, wherein the degree of alkoxylation n is from 1 to 30.

11. The compound of claim 10, wherein the degree of alkoxylation n is from 3 to 10 and (AO) is an ethylene oxide unit.

12. The method of claim 2, wherein the compound of formula (I) is copolymerized in the polymer.

13. The method of claim 2, further comprising initiating the reaction with potassium peroxodisulfate or a redox initiator.

14. The method of claim 2, wherein the compound of formula (I) is in salt form.
